(19) United States
(12) Patent Application Publication
(10) Pub. No.: US 2020/0388368 A1
(43) Pub. Date: Dec. 10, 2020

(54) SYSTEM AND METHOD FOR LEVERAGING ELECTRONIC HEALTH RECORD INFORMATION AND FUNCTIONALITY

(71) Applicant: Precision Medicine Group, LLC, Bethesda, MD (US)

(72) Inventors: Matthew Pitzel, Solon, OH (US); Tim Van Aken, Voorhees, NJ (US)

(21) Appl. No.: 16/876,650

(22) Filed: May 18, 2020

Related U.S. Application Data
(60) Provisional application No. 62/850,260, filed on May 20, 2019.

(51) Int. Cl. G16H 20/10; G16H 10/60; G16H 40/20

(52) U.S. Cl. CPC G16H 20/10; G16H 40/20; G16H 10/60

(57) ABSTRACT

A system and method of improving the workflow of healthcare providers assisting patients in reducing out-of-pocket expenses associated with medications by aiding healthcare providers in locating manufacturer provided resources including educational information, patient assistance programs, vouchers and/or co-pay cards and, automatically populating fields with the resource with data from an electronic health record database.

### TECHNICAL FIELD

The present disclosure relates generally to improving the workflow for healthcare providers in educating and assisting patients in filling prescriptions as well as reducing out-of-pocket expenses associated with medications, and more particularly to a system and method configured to assist healthcare providers in locating manufacturer provided education, patient assistance programs, vouchers, co-pay cards, or other beneficial patient resources and automatically filling in the information (if needed) necessary for completion with data from a patient's electronic health record.

### BACKGROUND

Advances in pharmaceuticals in recent times have led to the effective treatment of many disorders which only a few years ago had no available treatment. While these medications can be invaluable to those who need them, spending on prescription drugs continues to be one of the fastest-growing healthcare costs facing consumers.

With the high cost of drugs to individuals in out-of-pocket expenses, and with many alternative drug choices available, almost all patients are looking for better ways to understand the reason for the medication, fill their prescription, and afford and stay on medicines they have been prescribed. In an effort to address the pressure to lower prices for drugs and to create brand loyalty, many drug manufacturer companies now offer educational resources, patient assistance programs, vouchers, co-pay cards, as well as other patient resources.

Frequently physicians and other healthcare providers assist patients in locating these educational, streamlining, and cost reducing measures during medical visits. Unfortunately, the various patient resources are quite different among the manufacturer companies, which can lead to issues such as, e.g., patient frustration and longer processing times. Frequently the resources can be difficult to locate. Further, many of the resources require the often tedious manual entry of patient information. As a result, healthcare providers, who are already under pressure to minimize their non-reimbursable activities, view the process of locating and completing the necessary information as a burden, competing with the time they have available to treat other patients. As a result, many patients may not obtain the education and cost reduction to which they may otherwise be entitled from which they could benefit.

### SUMMARY OF THE DISCLOSURE

Embodiments of the present disclosure provide a system and method for improving the workflow of healthcare providers assisting patients in improving their education, streamlining the adjudication process, and reducing the out-of-pocket expenses associated with medicines by aiding healthcare providers in locating manufacturer provided resources including educational resources, patient assistance programs, vouchers, co-pay cards and other patient resources and, automatically populating selected resources with data from an electronic health record database.

One embodiment of the present disclosure provides a system for leveraging electronic health record information to improve a workflow of healthcare providers assisting patients in reducing out-of-pocket expenses associated with a prescribed medication. The system can include at least one healthcare provider computing device, an electronic health record server, and a base resource server. The at least one healthcare provider computing device can have a graphical user interface configured to aid a healthcare provider in location of the medication specific patient resource. The electronic health record server can be configured to collect and store patient information. The base resource server can be configured to store one or more medication specific patient resources and to establish communication between the at least one healthcare provider computing device and electronic health record server, and to automatically populate patient information from the electronic health record server into the medication specific patient resource as an aid in assisting patients in reducing out-of-pocket expenses associated with a prescribed medication.

In one embodiment, the medication specific patient resource is at least one of educational information, a patient assistance program, a discount voucher, or co-pay card. In one embodiment, the patient information includes at least one of a medical history, medications, allergies, immunizations status, laboratory test results, radiology images, vital signs, patient site, patient weight, demographic information, or billing information.

In one embodiment, the graphical user interface of the at least one healthcare provider computing device is configured to aid in guiding a healthcare provider workflow during patient visits. In one embodiment, the graphical user interface includes one or more healthcare provider selectable tabs configured to organize categories of healthcare provider workflows. In one embodiment, at least one of the one or more healthcare provider selectable tabs is installed on the graphical user interface via the base resource server.

In one embodiment, the graphical user interface includes at least one of a text box or drop-down menu configured for an entry of a prescription drug name. In one embodiment, upon entry of a prescription drug name, the graphical user interface is configured to display one or more associated medication specific patient resources. In one embodiment, the graphical user interface is configured to enable selection of one of the one or more associated medication specific patient resources stored on the base resource server. In one embodiment, the graphical user interface is further configured to request one or more signatures for completion of the medication specific resource. In one embodiment, the system further includes an internet-based server configured to collect a completed medication specific patient resource.

Another embodiment of the present disclosure provides a system for leveraging electronic health record information to improve a workflow of healthcare providers assisting patients in reducing out-of-pocket expenses associated with a prescribed medication. The system can include an Internet-based server, at least one healthcare provider computing device, an electronic healthcare record server, and a base resource server. The internet-based server can be configured to host a website on which a medication specific patient resource is provided. The at least one healthcare provider computing device can have a graphical user interface configured to aid a healthcare provider in location of the medication specific patient resource on the internet-based server. The electronic health record server can be configured to collect and store patient information. The base resource server can be configured to establish communication between the at least one healthcare provider computing device, electronic health record server, and internet-based server to enable automatic population of patient information from the electronic health record server into the medication specific patient resource as an aid in assisting patients in reducing out-of-pocket expenses associated with a prescribed medication.

Another embodiment of the present disclosure provides a method of leveraging electronic health record information to improve a workflow of healthcare providers assisting patients in reducing out-of-pocket expenses associated with a prescribed medication, comprising: accepting a desired therapy on a graphical user interface; displaying one or more medication specific patient resources associated with the desired therapy on the graphical user interface; accepting a selection of one of the one or more medication specific patient resources; obtaining the selected medication specific patient resource from at least one of a base resource server or an internet-based server; populating fields in the selected medication specific resource with patient information from an electronic health record server; and obtaining one or more signatures for completion of the selected medication specific resource.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a system architecture diagram depicting a system configured to improve the workflow of healthcare providers assisting patients in reducing out-of-pocket expenses associated with medicines, in accordance with an embodiment of the disclosure.

FIG. 2 is a flowchart depicting a method for improving the workflow for healthcare providers assisting patients in reducing out-of-pocket expenses associated with medicines, in accordance with an embodiment of the disclosure.

FIG. 3 is an exemplary graphical user interface screenshot depicting a selection of a desired patient therapy and one or more resources associated with a selected therapy, in accordance with an embodiment of the disclosure.

FIG. 4 is an exemplary graphical user interface screenshot depicting a selected resource located on a base resource server and automatically populated with data from an electronic health record, in accordance with an embodiment of the disclosure.

FIG. 5 is an exemplary graphical user interface screenshot depicting a selected resource located on a base resource server, where no data from the electronic health record is required to complete the resource, in accordance with an embodiment of the disclosure.

FIG. 6 is an exemplary graphical user interface screenshot depicting a selected resource calling for one or more signatures, consents or authorizations, in accordance with an embodiment of the disclosure.

FIG. 7 is an exemplary graphical user interface screenshot depicting options for providing one or more signatures, consents or authorizations, in accordance with an embodiment of the disclosure.

FIG. 8 is an exemplary graphical user interface screenshot depicting one step involved in an electronic signature, consent or authorization, in accordance with an embodiment of the disclosure.

FIG. 9 is an exemplary graphical user interface screenshot depicting a selected resource located on a third-party resource server, in accordance with an embodiment of the disclosure.

FIG. 10 is an exemplary graphical user interface screenshot depicting the selection of a tab to initiate a method for improving the workflow for healthcare providers assisting patients in reducing out-of-pocket expenses, in accordance with an embodiment of the disclosure.

FIG. 11 is an exemplary graphical user interface screenshot depicting a selected resource located on a base resource server and automatically populated with data from an electronic health record, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

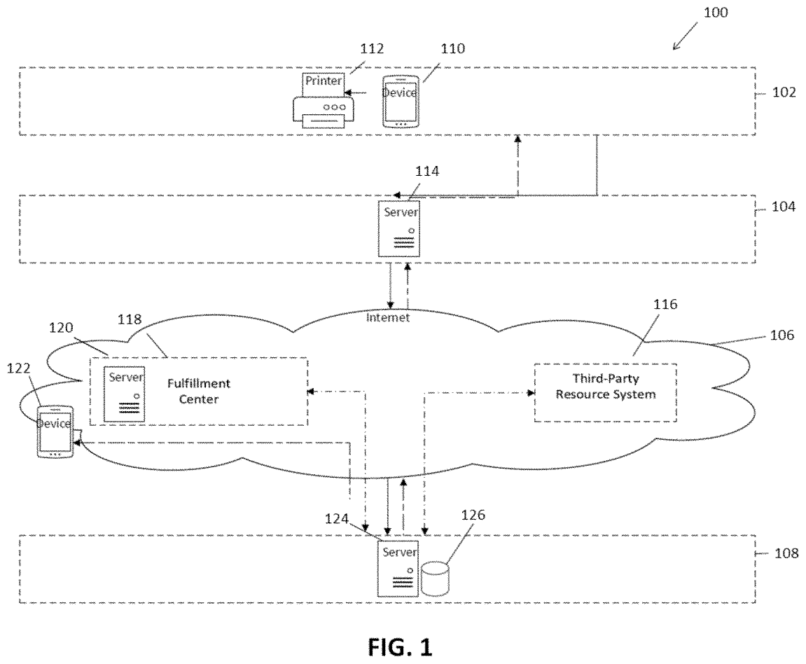

Referring to FIG. 1, an architecture diagram for a system 100 configured to improve the workflow of healthcare providers assisting patients in, e.g., reducing out-of-pocket expenses associated with medicines, is depicted in accordance with an embodiment of the disclosure. In one embodiment, the system 100 can generally include one or more on-site healthcare provider components 102, one or more electronic health record (EHR) components 104, one or more Internet-based components 106, and one or more base resource network components 108. Other system architecture designs are also contemplated.

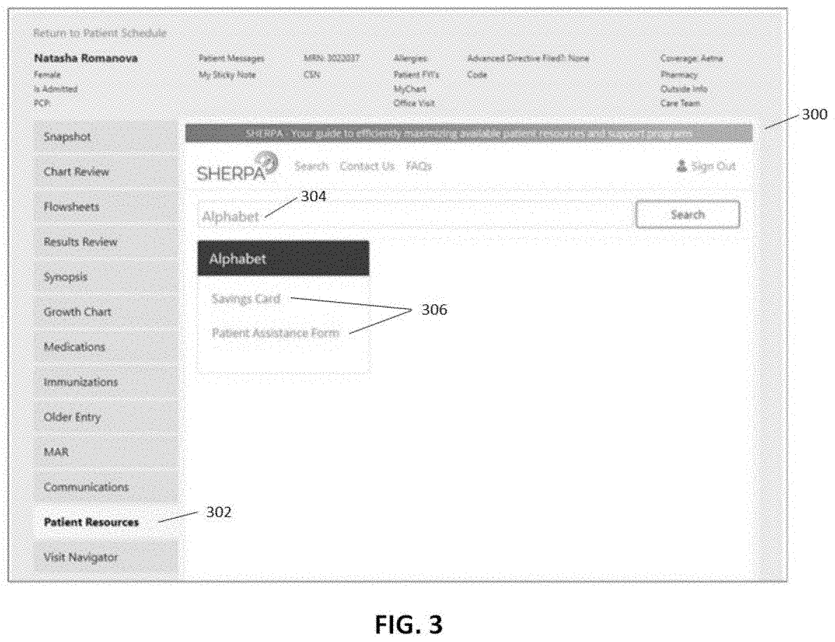

In one embodiment, the one or more on-site healthcare provider components 102 can, for example, include one or more computing devices 110 configured to be operated by a healthcare provider (e.g., physician, physician assistant, nurse, or any other provider with access to electronic health records). Examples of computing devices 110 include desktop computers, laptop computers, mobile computing devices, tablets, cellular telephones, and the like. The one or more computing devices 110 can include a user interface, such as, e.g., a graphical user interface 300 (as depicted in FIG. 3-8) configured to enable a healthcare provider to access the other components 104, 106, 108 of the system 100, thereby aiding in the location of manufacturer provided patient assistance programs, vouchers and/or co-pay cards, and where possible, automatically filling in the information necessary for completion with data gathered from the electronic health record components 104. In one embodiment, the one or more on-site healthcare provider components 102 can further include a printer 112 to enable the healthcare provider to print certain aspects of the graphical user interface and/or other information available within the system 100.

The one or more electronic health record components 104 can include a server 114 configured to collect and store patient information. Examples of patient information include a medical history, medications and allergies, immunization status, laboratory test results, radiology images, vital signs, personal statistics such as age, height and weight, demographic information and billing information. In some embodiments, the server 114 can be co-located with the on-site healthcare provider components 102. In other embodiments, the server 114 can be remotely located, and accessible via a wired or wireless network.

The Internet-based components 106 can include a third-party resource server 116, such as a server providing a drug manufacturer or pharmaceutical company website, where for example one or more medication specific patient resources (e.g., educational information, assistance programs, vouchers and/or co-pay cards) can be located. The Internet-based components 106 can further include a manufacturer company fulfillment center 118, which may include a server 120 configured to collect completed patient assistance programs, vouchers and/or information from patient co-pay cards. In one embodiment, the Internet-based components 106 can further include, or be in communication with, one or more other computing devices 122, thereby enabling healthcare providers utilizing the system 100 to communicate electronically with patients and other individuals.

The base resource network components 108 can include a server 124 and a database 126 configured to tie the various other system components 102, 104, 106 together such that they may be in communication with one another in various combinations and configurations, and to store certain manufacturer provided resources (e.g., educational information, or one or more forms relating to patient assistance programs, vouchers and/or co-pay cards), for ease in automatically populating patient information from the electronic health record server 114 into the resource. Server 124 can communicate with the other system components via the Internet.

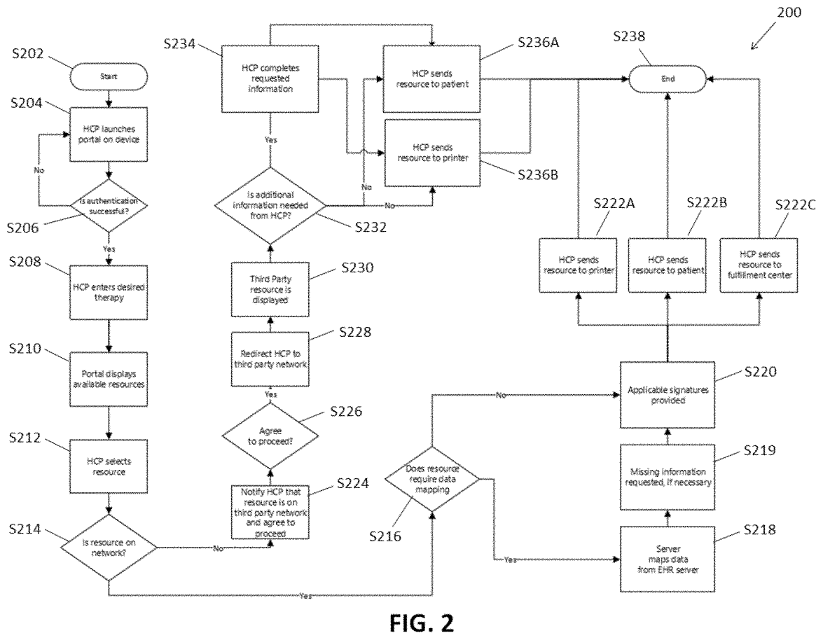

Referring to FIG. 2, a method 200 for improving the workflow for healthcare providers assisting patients in reducing out-of-pocket expenses associated with, e.g., prescription drugs is depicted in accordance with an embodiment of the disclosure. The method begins at S202. At S204, a healthcare provider launches a portal on the computing device 110. In some embodiments, the portal displays a login screen for authentication prior to enabling access to the healthcare record system. At S206, verification of the login information or other credentials provided is completed.

If authentication is successful, a healthcare provider can enter the patient name to display certain patient information from the patient electronic health record. For example, with reference to FIG. 3, an exemplary screenshot from a graphical user interface 300 is depicted in accordance with an embodiment of the disclosure. Collectively, exemplary screenshot depicted in FIG. 3 and other screenshots disclosed hereafter represent portions of a user interface, such as the graphical user interface 300, which may be displayed on the healthcare provider computing device 110. As depicted, the fictitious patient name “Natasha Romanovo” has been entered. In one embodiment, information, such as the patient's medical record number (MRN) and other patient identification can be displayed (e.g., in proximity to a top of the screenshot).

The graphical user interface 300 can be utilized by a healthcare provider as an aid or tool in guiding the workflow during patient visits. For example, during a visit, a healthcare provider can select various tabs located in the graphical user interface 300 during the process of administering care to the patient. As depicted in FIG. 3, in one non-limiting example, the tabs can include names such as: snapshot; chart review; flow sheets, results review, synopsis, growth chart, medications, immunizations, older entry, MAR, communications, and visit navigator. The inclusion of other tabs or combinations of tabs is also contemplated. Any number and combination of tabs with various names and functions may be used. In addition, other mechanisms besides tabs may be used to organize various categories to guide a user in the selection process, whether via the graphical user interface shown in FIG. 3, a different graphical user interface having another configuration, a non-graphical user interface (e.g., audio, alphanumeric, etc.) or any combination thereof.

In one embodiment, a user, such as a healthcare provider, can access the base resource server 124 via the computing device 110 to load an additional tab 302, depicted in FIG. 3 as a tab named “patient assistance” (although other tab names such as “patient resources,” “ resource portal,” or the like are also contemplated). For example, in one embodiment, the additional tab 302 can be added by downloading software configured to execute the method 200 via Epic's App Orchard store, or a similar site for downloading software applications. Once downloaded, tab 302 may be installed and added to the previously existing graphical user interface 300. Thereafter, the newly added tab 302 can be selected to complete method 200.

With continued reference to both FIGS. 2 and 3, at 5208, the healthcare provider can enter a desired therapy in, e.g., a text box 304. The desired therapy can include, for example, a patient disease or condition, a treatment plan, a manufacturer or pharmaceutical company name, a medication, supplement or prescription drug name, or the like. In some embodiments a drop-down menu or the like can be provided to display available resources from which a healthcare provider can select the appropriate therapy. Alternatively, a healthcare provider may simply type in or otherwise request (e.g., via a voice command or the like) the selected therapy. As depicted in FIG. 3, the fictitious drug name “Alphabet” has been entered.

With the desired therapy entered (e.g., Alphabet), at 5210 the user interface 300 can display one or more resources 306 available for the selected therapy. Such resources can include, for example, savings cards, patient assistance enrollment, and other resources that help to educate or reduce the cost of medications. At S212, the healthcare provider can select one of the displayed resources 306. When one of the resources 306 is selected, at S214 a determination can be made by the base resource server 124 as to whether the resource is stored within the base resource network components 108 (i.e., the base resource server 124 or associated database 126) or whether the resource is retained with a third-party resource server 116.

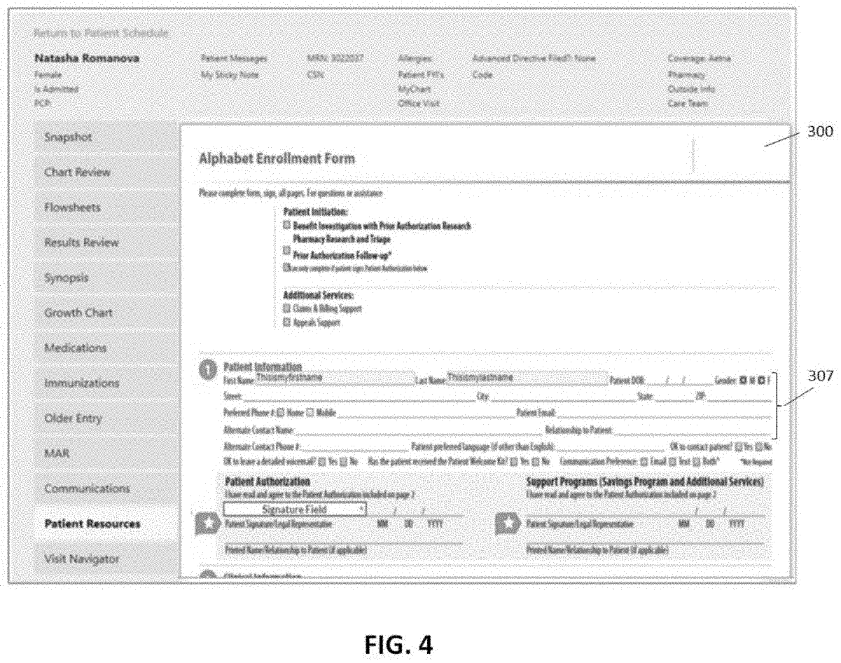

If the resource 306 is stored within the base resource network components 108, the method proceeds to S216, where the inquiry is made as to whether the resource requires data mapping from the electronic health record server 114. For example, with reference to FIG. 4, if the “patient assistance form” resource is selected (which in this embodiment is stored within the base resource network components 108 and requires data mapping), at S218 the base resource server 124 automatically populates the resource fields 307 with patient information from the electronic health record server 114. For example, in one embodiment, the server 124 can automatically enter the patient's name, date of birth, gender, address, phone number and email address into the form with data obtained from the electronic health record. In other embodiments, more or less patient information can be entered, in various combinations, depending on the requirements of a particular form and how much of the information required by the form can be obtained from the electronic health record. At S219, any additional information, which may be missing or otherwise not included in the patient's electronic health record, can be requested and/or filled in via the user interface 300. In embodiments, the additional information can be requested by a pop-up window or message having data entry boxes requesting the required information.

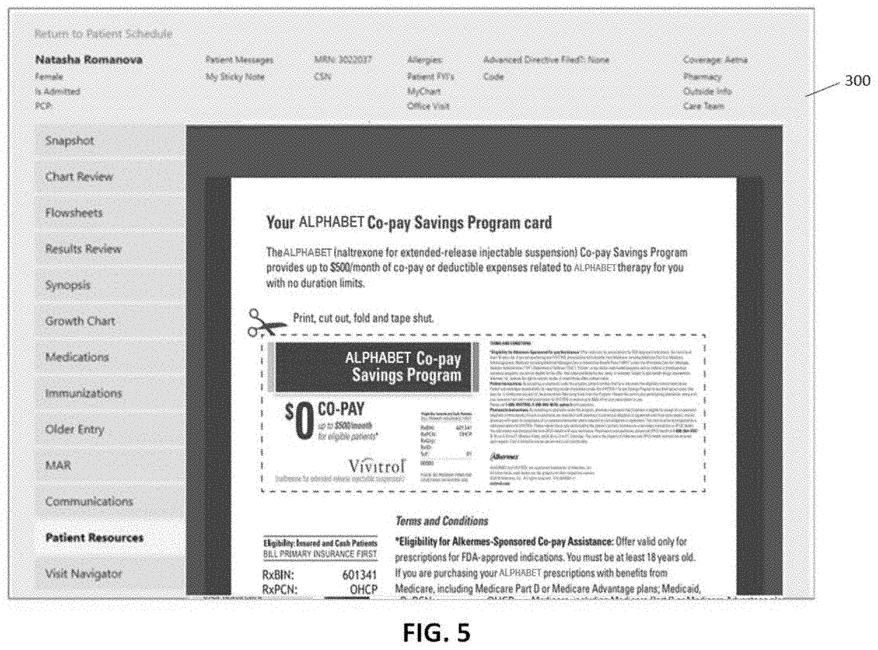

At S220, the resource can then be presented on the computing device 110, with all of the required information fields filled in with the applicable patient data. Alternatively, where patient no data is required the method 200 can proceed from S216 directly to S220 for presentation of the resource on the computing device 110. For example, with reference to FIG. 5, if the fictitious drug name “Alphabet” and the co-pay savings program card is selected, a copy of the resource can be displayed at S220. Alternatively, the resource may be filled out without presenting the completed form to the user.

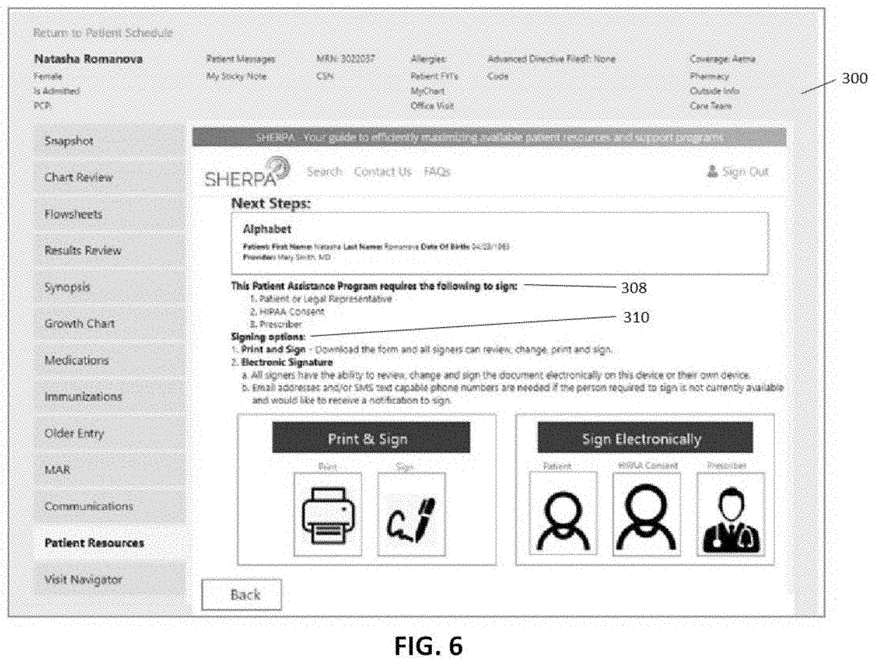

At S220, one or more signatures and/or authorizations can be requested. For example, with reference to FIG. 6, in one embodiment, the user interface 300 can indicate the type of signatures and/or authorizations required 308, as well as the options 310 for providing the signatures and/or authorizations. In some embodiments, the signatures and/or authorizations can include a signature from the patient or legal representative of the patient, Health Insurance

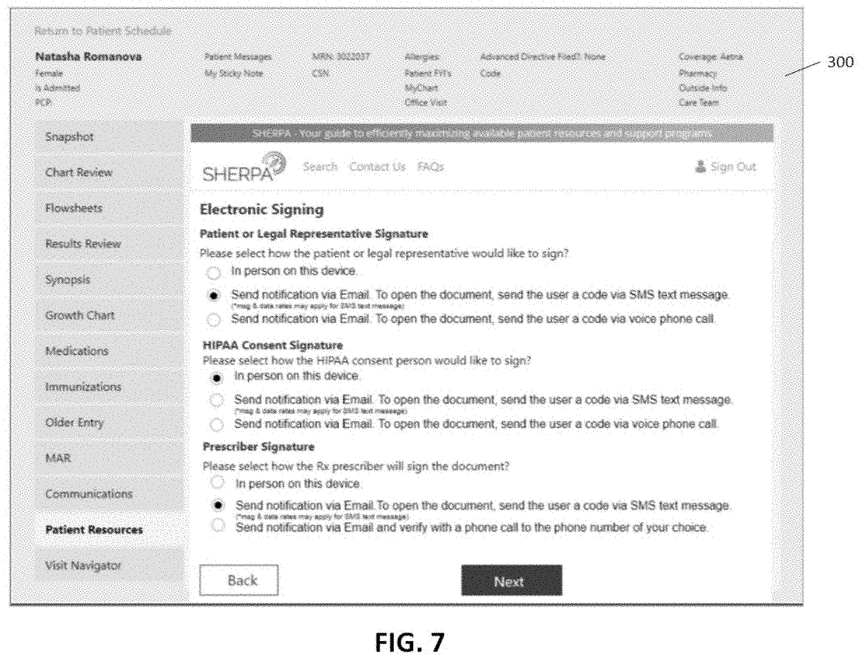

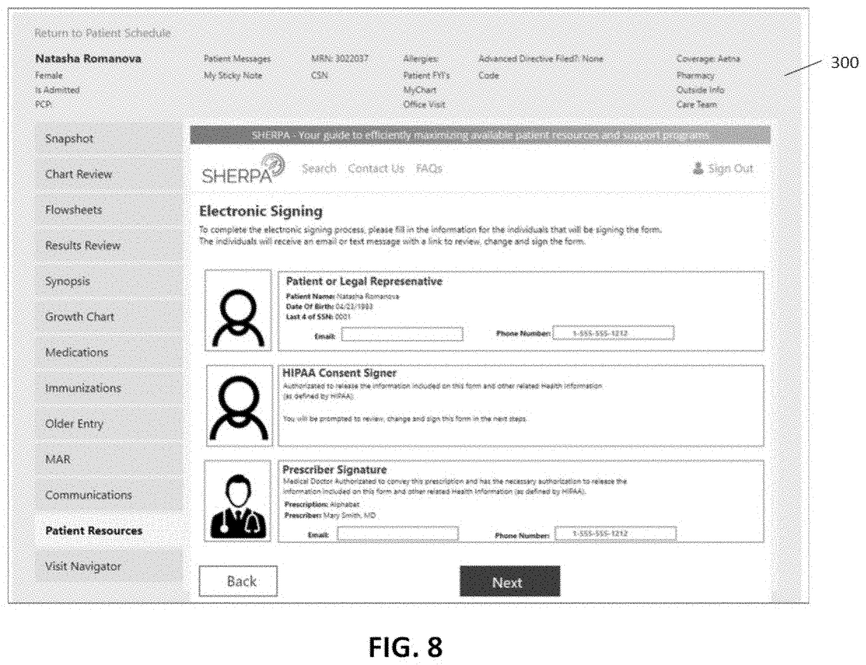

Portability and Accountability Act (HIPPA) consent, a prescriber signature, and the like; although other types of signatures, authorizations and consents are also contemplated. In some embodiments, the signing options can include downloading the form to enable a printed document to be physically signed, or the ability to electronically sign the resource. With reference to FIG. 7, in some embodiments, the user interface 300 can provide a number of different ways in which the resource can be electronically signed. For example, as depicted, the electronic signature and/or consent can be completed through the user interface 300 (e.g., by capturing a signature on a touchscreen of the computing device 110), or via e-mail; other methods of electronic signature are also contemplated. Where the electronic signature, authorization and/or consent is to be completed via e-mail, various authentication options can be provided. For example, in some embodiments, a code sent via SMS, text message or via phone, may be required to complete the electronic signature, consent and/or authorization. With reference to FIG. 8, in some embodiments, the user interface 300 can request relevant contact information (e.g., e-mail address, phone number, etc.) necessary to complete the resource. With continued reference to the method 200 flowchart of FIG. 2, at S222A-C, the healthcare provider can optionally select whether to: (1) send the resource to the printer 112; (2) send the resource to a patient computing device 122; and/or (3) send the resource to a fulfillment center 118. In some embodiments, the resource can be sent to the fulfillment center, patient or other destination via email, fax, or the like.

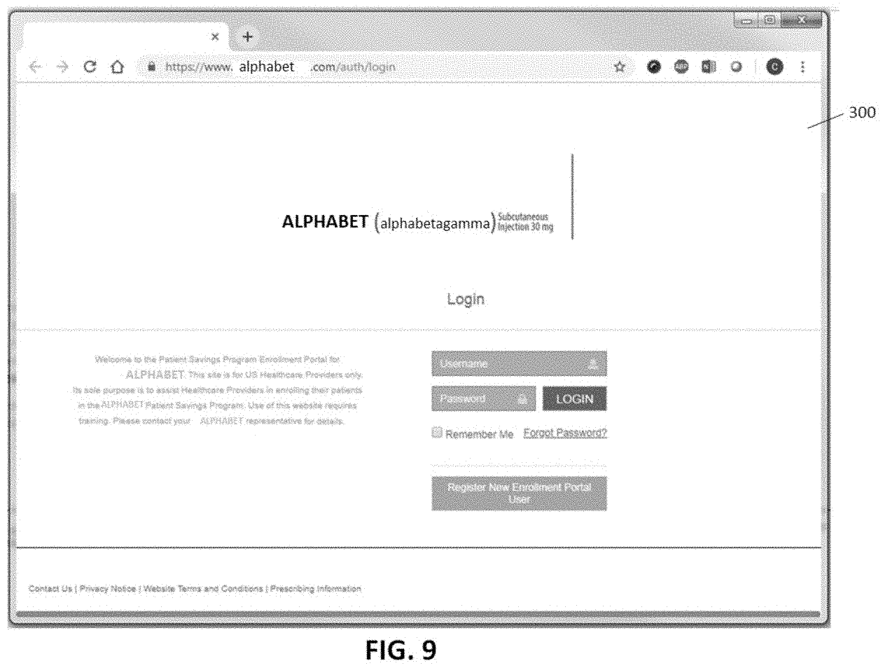

Alternatively, if it is determined by the base resource server 124 that the resource 306 is not stored within the base resource network components 108, at S224 the healthcare provider is notified that the resource 306 is located on a third-party resource server 116. At S226, a determination is made as to whether the healthcare provider agrees to proceed to the third-party resource server 116. If the healthcare provider agrees to proceed, at S228 the graphical user interface 300 provides a portal to the third-party resource server 116. For example, with reference to FIG. 9, if the “savings card” resource is selected (which in this embodiment is stored on a third-party resource server 116), at S230 the resource from the third-party resource server 116 is displayed.

At S232, a determination can be made as to whether additional information is needed to complete the resource. If additional information is required, at S234 the healthcare provider can complete the requested information. At S236A-B, the healthcare provider can optionally select whether to, for example: (1) send the resource to a patient computing device 122; and/or (2) send the resource to the printer 112. At S238, the method 200 is complete with the patient having been assisted in filling out the resource and/or submitting the resource to the fulfillment center.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

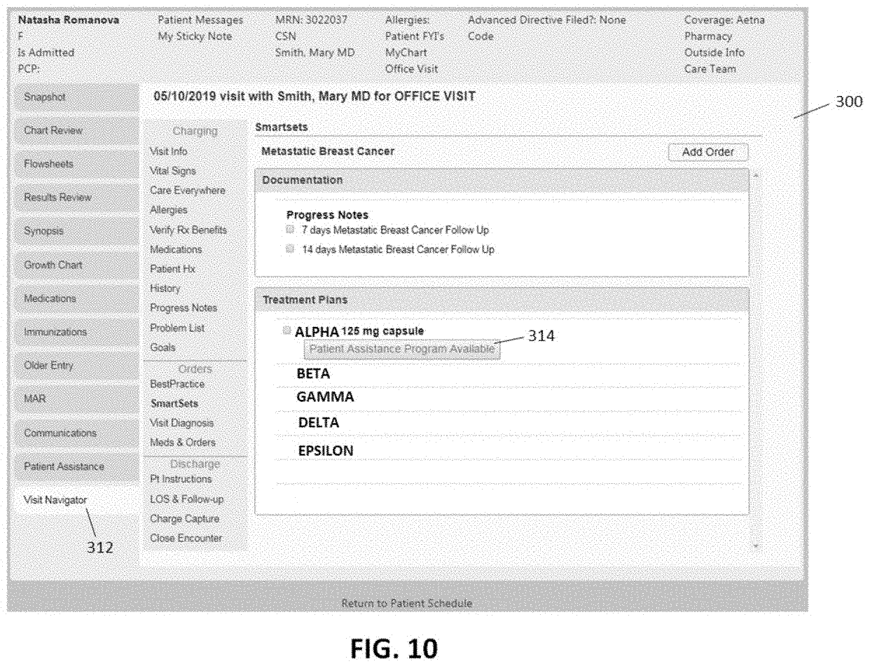

With reference to FIG. 10, the visit navigator tab 312 on the graphical user interface 300 can alternatively and/or additionally be utilized to access patient assistance resources. After selecting the visit navigator tab 312, a healthcare provider can then select the treatment plan for ordering a prescription medication. In one non-limiting embodiment, a selection box 314 can be provided under the medication indicating whether a patient assistance program; the listing of resources, such as patient education, co-pay card, voucher, or other patient resource is available. Selection of the selection box 314 can initiate portions of the method 200.

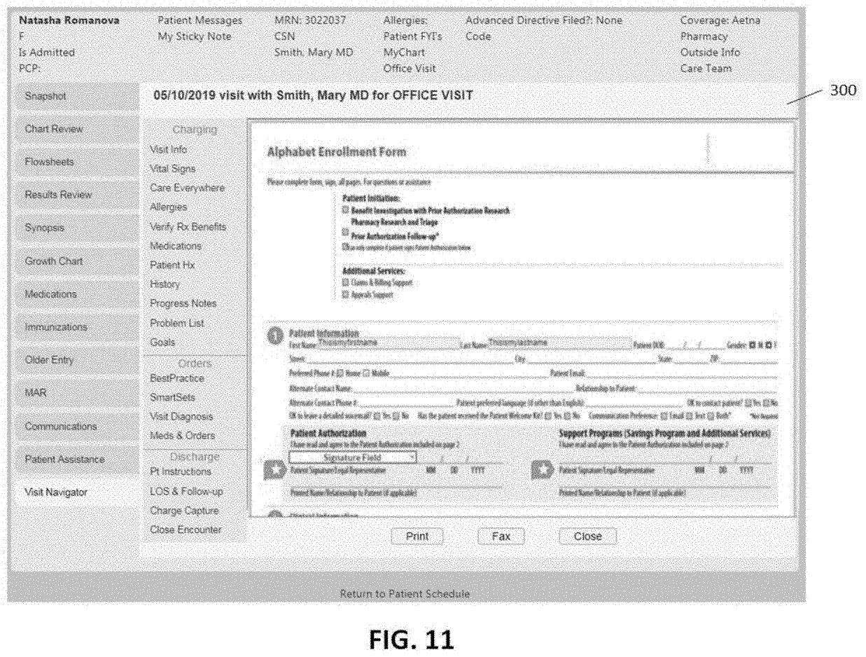

For example, with reference to FIG. 11, selection of the selection box 314 can be followed by S214, wherein a determination is made as to whether the resource is stored within the base resource network components 108 or whether the resource is retained with a third-party resource 116. As depicted in FIG. 8, if the resource is located on the base resource network components 108, method 200 can proceed through steps S216-S220, to populate the fields with patient information from electronic health records, and obtain any required signatures. If the resource is located on a third party network, the method 200 can proceed through steps S224 to S236A-B to provide access to the resource via the third party network.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A system for leveraging electronic health record information to improve a workflow of healthcare providers assisting patients in reducing out-of-pocket expenses associated with a prescribed medication, the system comprising:
at least one healthcare provider computing device having a graphical user interface configured to aid a healthcare provider in location of a medication specific patient resource;
an electronic health record server configured to collect and store patient information; and
a base resource server configured to store one or more medication specific patient resources and to establish communication between the at least one healthcare provider computing device and the electronic health record server, wherein the base resource server is configured to automatically populate patient information from the electronic health record server into the one or more medication specific patient resources as an aid in assisting patients in reducing out-of-pocket expenses associated with a prescribed medication.

2. The system of claim 1, wherein the one or more medication specific patient resources include at least one of educational information, a patient assistance program, a discount voucher, or a co-pay card.

3. The system of claim 1, wherein the patient information includes at least one of a medical history, medications, allergies, immunizations status, laboratory test results, radiology images, vital signs, patient height, patient weight, demographic information, or billing information.

4. The system of claim 1, wherein the graphical user interface of the at least one healthcare provider computing device is configured to aid in guiding a healthcare provider workflow during patient visits.

5. The system of claim 1, wherein the graphical user interface includes one or more healthcare provider selectable tabs configured to organize categories of healthcare provider workflows.

6. The system of claim 5, wherein at least one of the one or more healthcare provider selectable tabs is installed on the graphical user interface via the base resource server.

7. The system of claim 1, wherein the graphical user interface includes at least one of a text box or drop-down menu configured for an entry of a prescription drug name.

8. The system of claim 7, wherein upon entry of a prescription drug name, the graphical user interface is configured to display one or more associated medication specific patient resources corresponding to the prescription drug name.

9. The system of claim 1, wherein the graphical user interface is configured to enable selection of one of the one or more associated medication specific patient resources stored on the base resource server.

10. The system of claim 1, wherein the graphical user interface is further configured to request one or more signature for completion of the medication specific resource.

11. The system of claim 1, further comprising an internet-based server configured to collect a completed medication specific patient resource.

12. A system for leveraging patient electronic health record information to improve a workflow of healthcare providers assisting patients in completion of a medication specific patient resource to reduce out-of-pocket expenses associated with a prescribed patient therapy, the system comprising:
a base resource server configured to receive a request for one or more medication specific patient resources related to a prescribed patient therapy, determine an existence of one or more medication specific patient resources related to the prescribed patient therapy, provide a listing of the one or more medication specific patient resources for display, receive a selection of one medication specific patient resource of the one or more medication specific patient resources, populate one or more fields in the selected medication specific patient resource with electronic health record information for the patient, and request one or more signatures for completion of the medication specific patient resource as an aid in assisting patients in reducing out-of-pocket expenses associated with the desired patient therapy.

13. The system of claim 12, wherein the medication specific patient resource is at least one of educational information, a patient assistance program, a discount voucher, or co-pay card.

14. The system of claim 12, further comprising a graphical user interface configured to aid in guiding a healthcare provider workflow during patient visits.

15. The system of claim 14, wherein the graphical user interface includes one or more healthcare provider selectable tabs configured to organize categories of healthcare provider workflows.

16. The system of claim 12, wherein the graphical user interface includes at least one of a text box or drop-down menu configured for an entry of a prescription drug name.

17. The system of claim 16, wherein upon entry of a prescription drug name, the graphical user interface is configured to display one or more associated medication specific patient resources.

18. The system of claim 12, wherein the graphical user interface is configured enable selection of one of the one or more downloadable medication specific patient resources.

19. The system of claim 12, wherein the graphical user interface is configured to request an electronic signature for completion of the medication specific patient resource.

20. A method of leveraging patient electronic health record information to improve a workflow of healthcare providers assisting patients in completion of a medication specific patient resource to reduce out-of-pocket expenses associated with a prescribed patient therapy, the method comprising:
receiving e a request for one or more medication specific patient resources related to a prescribed patient therapy,
determining an existence of one or more medication specific patient resources related to the prescribed patient therapy;
providing a listing of the one or more medication specific patient resources for display;
receiving a selection of one medication specific patient resource of the one or more medication specific patient resources;
populating one or more fields in the selected medication specific patient resource with electronic health record information for the patient; and
requesting one or more signatures for completion of the medication specific patient resource as an aid in assisting patients in reducing out-of-pocket expenses associated with the desired patient therapy.
